(19) United States
(12) Patent Application Publication
(10) Pub. No.: US 2005/0191326 A1
(43) Pub. Date: Sep. 1, 2005

(54) Materials and methods for creating customized compositions having a temporary visual indicator

(76) Inventor: Richard J. Melker, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/788,541

(22) Filed: Feb. 27, 2004

(51) Int. Cl. A61K 7/42; C08K 5/48

(52) U.S. Cl. 424/401; 524/90

(57) ABSTRACT

The present invention relates to novel compositions, which provide an indicator for location and/or concentration of the composition during application. In a preferred embodiment, the indicator is a compound that is visible at a first pH and invisible at a second pH. The composition includes the indicator and a volatile base or acid. The volatile base or acid enables the composition to be at a first pH so that the indicator is visible when first applied to a desired surface. However, upon evaporation or degradation of the volatile acid or base, the composition will be at a second pH, causing the indicator to disappear.

### BACKGROUND OF INVENTION

There are many situations in which it is desirable to know where a particular material is being applied or has previously been applied.

For example, in medicine it is often necessary to ascertain whether surgical scrub has been applied to an area of skin to be disinfected. Current surgical scrubs contain iodine as a disinfecting agent. In addition to acting as a disinfectant, the iodine in the scrub stains the skin on contact. Thus, the iodine serves the dual function of a color guide for application and of a disinfecting agent to destroy, neutralize, or inhibit the growth of disease-carrying microorganisms.

Iodine, however, has fallen into disfavor for use in surgical scrubs due to recent studies that have found the growth of certain bacterial pathogens in iodine. See Mertz PM, et al. “A new in vivo model for the evaluation of topical antiseptics on superficial wounds. The effect of 70% alcohol and povidone-iodine solution,” Arch Dermatol., 120(1):58-62 (1984). Additionally, iodine has been found to be toxic to dermal cells, thereby impeding the healing of surgical incisions. See Smoot EC 3rd, et al. “In vitro toxicity testing for antibacterials against human keratinocytes,” Plast Reconstr Surg. 87(5):917-24 (1991). Newer antiseptic scrubs/disinfecting agents such as benzalkonium chloride are colorless and concern has been expressed by the medical profession that areas requiring disinfection are being missed because the antiseptic scrub cannot be visualized. Thus, there exists a need for a means to temporarily color the antiseptic so that it is applied to the proper areas.

Other situations in which a temporary visible indicator would be useful include, and are not limited to, inks for writing or printing, clear lacquers, varnishes, or sprays; pesticides, herbicides, or fertilizers; topical formulations (i.e., lotions, creams, gels and/or sprays); cleaning solutions; protective sealants (i.e., carpet or fabric protective sealants); polish or wax solutions for vehicles (i.e., car, boat); and the like. With all of these materials, it is advantageous, or critical, to know precisely where the material is being applied or has previously been applied. Unfortunately, these materials often do not include a means for helping the user differentiate areas to which material application is being applied or has already been accomplished.

An adhesive compound which undergoes color changes upon application has previously been described (U.S. Pat. No. 4,954,544). The indicator affecting the color change in the adhesive also serves to enhance the physical characteristics of the adhesive compound by contributing to improved flow and bonding. The indicator also adds to the economy of the product by allowing for formulations in which less adhesive compounds are required, while still imparting improved flow and bonding. This indicator, however, does not solely provide the function of being a visual guide for application of the material to a surface.

It is already well-known, as exemplified in U.S. Pat. Nos. 5,532,029 and 5,548,010, that paints can be provided that change color with time so that at the time of application, the location of the applied paint is presented. The U.S. Pat. No. 5,548,010 discloses a paint that changes color as a result of a light-unstable dye that is mixed with the paint. The light-unstable dye provides a secondary color to the paint, which dissipates over time.

Further, U.S. Pat. No. 6,120,949 discloses the addition of a light-stable colorant for paint that is mutable when exposed to a specific, narrow band-width radiation, such as ultra-violet (UV) radiation. The colorant can be added to paint compositions for application to a surface. The colorant will present a specific color until presentation with UV irradiation to irreversibly mutate the color to become substantially colorless. UV radiation, however, can be hazardous to health. For example, UV radiation exposure can cause erythema, photoaging, skin cancer, and photokeratitis.

In a related application, U.S. patent application Ser. No. 2003/0191036 discloses a soap having properties (i.e., color, viscosity, smell, temperature) the change in a specific period of time to notify the user that the time has passed. In particular, the use of a food dye in an antibacterial soap with ascorbic acid and iron chloride in a “decolorizing agent” are described in the application as a means for changing the soap from a green color to a blue color, depending on the concentration of the decolorizing agent.

Also, U.S. Pat. Nos. 6,139,821; 5,997,891; 5,837,645; and 5,523,075 disclose compositions, in particular sunscreens, in which a pH-dependent indicator is included, where the indicator is visible at a first pH and invisible at a second pH.

Generally, the above-described adhesives, paints, and other compositions are mass manufactured and provided to the user with the temporary indicator already incorporated in the materials. Thus, the user cannot adjust the amount of temporary visible indicator to be added and/or adjust the period of time in which the indicator would appear or eventually disappear. Further, because these materials are mass-manufactured with the temporary indicator already incorporated in the materials, material selection may be limited and with limited selection, prices may be inflated. Moreover, the user is obligated to purchase items, such as the above-noted paint, that may not fully address the user's needs (i.e., unwanted paint colors) or are potentially harmful to the user's health (i.e., requirement of UV radiation to induce color change). Heretofore there has not been devised a safe and easy method for users to prepare customized materials to be applied to a surface, having a temporary visible indicator that enables the user to establish the location, duration of visible indication, and/or concentration of the materials after application.

### BRIEF SUMMARY OF THE INVENTION

The present invention provides novel materials and methods for preparing compositions having a temporary visual indicator customized to an individual user's needs, where the individual user adds a temporary indicator to a selected material so that the location, duration of visible indication, and/or concentration of the material after application to a surface can be determined by the individual user.

The subject invention enables the user to make, at a minimal monetary and time expense to the user, customized materials that are temporarily discernible after application. According to the subject invention, methods for making customized materials for application to a surface comprise the steps of selecting a material and a temporary visual indicator, and adding the temporary visual indicator to the material to form a customized composition. The customized composition can then be applied to a desired surface, wherein the indicator is temporarily detectable by the user. The temporary visual indicator identifies to the user (or others) the location and/or concentration of the applied material and eventually becomes undetectable so that the material functions as intended.

The user may desire a material that is visibly discernible for a specified period of time (i.e., a wax product that is originally colorless but upon application to a surface, is visible to the user for a specified period of time to ensure adequate surface treatment, and eventually returns to the original colorless property so that the wax functions as intended). Accordingly, in certain embodiments of the invention, a user may customize a material to include a temporary visible indicator that is discernible for a specified period of time.

The temporary visual indicators that can be used according to the subject invention are well-known to the skilled artisan. In accordance with the present invention, contemplated temporary visual indicators for admixture with materials to be applied to a desired surface include, but are not limited to, Basonyl® green NB-832 (triarylmethane) (BASF Corp., Rensselaer, N.Y.), Basonyl® Blue-650 (triarylmethane) (BASF Corp., Rensselaer, N.Y.), “Flexo Yellow” 110 LD (diarylmethane) (BASF Corp., Rensselaer, Nyf); FD&C#2 Blue Powder (indigotene) (Hilton-Davis, Co., Cincinnati, Ohio), FD&C#2 AL. Lake, FD&C#2 (indigotene) (Triton Colors, Inc., Elmwood Park, N.J.), Pylam® Blue LX 5595 (triarylmethane) (Pylam Products Co., Inc., Garden City, N.Y.), FD&C Blue #1 Powder (triphenylmethane) (Warner-Jenkinson Co., Inc., St. Louis, Mo.), FD&C#1 Al.Lake (Warner-Jenkinson Co., Inc., St. Louis, Mo.), FD&C Yellow #5 (pyrozoine) (Warner-Jenkinson Co., Inc., St. Louis, Mo.), FD&C Green #3 (triphenylmethane) (Warner-Jenkinson Co., Inc., St. Louis, Mo.), Erythrosine Lake #9301 (Warner-Jenkinson Co., Inc., St. Louis, Mo.), FD&C Yellow Lake #5 (Warner-Jenkinson Co., Inc., St. Louis, Mo.); phenolphthalein, 3,3-bis[4-hydroxyphenyl]-1-[3H]isobenzofuranone; bromthymol blue; thymol blue; phenol red; cresol red; m-cresol purple; methyl violet; methyl orange; bromocresol green; methyl red; thymolphthalein; and alizarin yellow.

Advantageously, the temporary indicator provided according to the present invention is discernible only temporarily and disappears within a set period of time or as a result of user action (i.e., addition of chemicals to cause a change in visibility), and does not to interfere with the nature of the materials to be applied/used.

In a preferred embodiment, the temporary visual indicator is visible at a first pH level and is invisible at a second pH level. Accordingly, the indicator can be combined with a pH-modifying substance that establishes a first pH of the composition and which degrades, evaporates or otherwise effects a change in the pH to a second pH. Thus, the indicator is visible at the first pH and no longer visible at the second pH (after evaporation, degradation, etc. of the pH-modifying substance).

A pH-modifying substance may be combined with the indicator either prior to, during, or after the indicator has been added to the material selected for customization, in accordance with the present invention. Alternatively, a pH-modifying substance may be added to the selected material prior to addition of the indicator.

In particular embodiments, the period of time during which a temporary visual indicator is discernible can be manipulated by the individual user. For example, where a temporary visible indicator that is sensitive to pH is mixed with a selected material, the amount and/or concentration of a pH-modifying substance can be used to customize the period of time in which the indicator is visible to the individual user after application of the mixture to a desired surface (or other location).

In one embodiment, the temporary visual indicator can be added to a selected material with other components that affect the visibility and/or duration of visibility to the user. For example, oxidizing or reducing agents may be added to a customized composition to affect the visibility of the indicator, i.e., to increase the intensity of the indicator color and/or to make the indicator color dissipate in minutes, hours, or days.

As described herein, materials for customization in accordance with the present invention are produced by a user. Such materials are customized to include a temporary visual indicator based on the user's specifications. After application, the location and/or concentration of the customized compositions comprising the temporary visual indicator can be easily ascertained by the user. For example, in the case of sunscreen, the user selects any brand, type, and/or sun-protection factor (SPF) to be applied to skin, and adds a temporary visual indicator. In one embodiment, the amount of temporary visual indicator is commensurate with the duration of indicator visibility.

In one embodiment, the user adds a temporary visual indicator to materials to be applied to a desired surface, wherein the visual indicator is detectable at a first pH and invisible at a second pH. For example, the user can select a paint, and add to the paint a temporary visual indicator and a volatile base or acid, wherein the indicator is visible upon application to a desired surface while the volatile base or acid is present in the paint. A change in pH of the paint after the volatile base or acid evaporates or degrades causes the indicator to no longer be visible. The visual indicator can be reactivated to its visible form by temporarily restoring the pH of the applied paint to the first pH.

In a preferred embodiment, a user can customize materials for application to a surface, wherein the user selects a material; and phenolphthalein and a volatile base are mixed with the selected material. Contemplated volatile bases include, but are not limited to, ammonia, dimethylamine, trimethylamine, such that customized compositions of the invention have a pH greater than 9.0 when applied, but become neutral after a short period of time as a result of the evaporation or degradation of the volatile base.

The present invention also provides novel compositions that are useful for providing temporary visual indication of the location to which a material has been applied. The present invention provides materials (hereinafter referred to as “non-paint materials”) to be applied to a desired surface (i.e., materials for topical application to skin, materials for application to vehicles, materials for agricultural application) that include a light unstable dye. Paints are excluded from this particular embodiment of the invention, as are compositions related to paints (i.e., sealers, varnishes, lacquers). The light unstable dye enables the user to know the precise location where a material has been applied. The visibility of the dye dissipates as a result of oxidation, reduction, exposure to light, or combination thereof.

Preferred compositions of the invention comprise a light unstable dye and a component selected from the group consisting of: topical materials for application to human or animal skin, herbicides, fertilizers, pesticides, materials for application to vehicular surfaces, materials for use in cleaning, and protectants. Preferred light unstable dyes include: Basonyl® green NB-832 (triarylmethane) (BASF Corp., Rensselaer, N.Y.), Basonyl® Blue-650 (triarylmethane) (BASF Corp., Rensselaer, N.Y.), “Flexo Yellow” 110 LD (diarylmethane) (BASF Corp., Rensselaer, Nyf); FD&C#2 Blue Powder (indigotene) (Hilton-Davis, Co., Cincinnati, Ohio), FD&C#2 AL. Lake, FD&C#2 (indigotene) (Triton Colors, Inc., Elmwood Park, N.J.), Pylam® Blue LX 5595 (triarylmethane) (Pylam Products Co., Inc., Garden City, N.Y.), FD&C Blue #1 Powder (triphenylmethane) (Warner-Jenkinson Co., Inc., St. Louis, Mo.), FD&C#1 Al.Lake (Warner-Jenkinson Co., Inc., St. Louis, Mo.), FD&C Yellow #5 (pyrozoine) (Warner-Jenkinson Co., Inc., St. Louis, Mo.), FD&C Green #3 (triphenylmethane) (Warner-Jenkinson Co., Inc., St. Louis, Mo.), Erythrosine Lake #9301 (Warner-Jenkinson Co., Inc., St. Louis, Mo.), and FD&C Yellow Lake #5 (Warner-Jenkinson Co., Inc., St. Louis, Mo.).

In accordance with the present invention, a temporary visual indicator can be formulated with any materials that are applied to a surface, requiring indication of location and/or concentration of the applied material.

### DETAILED DISCLOSURE OF THE INVENTION

The present invention provides novel methods and compositions useful for providing a temporary visual indication of the location, concentration, and/or time period of treatment of an applied material. In particular, the present invention provides novel methods for customizing materials to include a temporary visual indicator.

In accordance with the present invention, a temporary visual indicator is any substance that is visible for a period of time as desired by a user (but that will eventually become invisible/colorless). The temporary visual indicator, when mixed with a selected material for application to a surface, preferably does not adversely affect the nature of the material. A temporary visual indicator can be in the form of a solid, liquid, or gas. For example, the temporary visual indicator can be in the form of a liquid, powder, a gel, capsules, or any form that can readily be added to materials that are to be applied to a desired surface.

The term surface, as contemplated herein, refers to an outer boundary of an object. Non-limiting examples of surfaces to which materials of the invention are applied include, but are not limited to, walls, floors, skin, vehicular exterior and interior surfaces (i.e., automobile, boats, trains, airplanes), furniture, appliances, glass, woods, plastics and other synthetic materials, metals, bodily organs (including skin, hair, nails, teeth, internal organs), leathers, papers, canvases, and mirrors.

In one embodiment, a method for making customized materials for application to a desired surface includes: selecting a material for application to a surface; selecting a temporary visual indicator; and mixing the temporary visual indicator into the material to form a customized composition. The customized composition can then be applied to the surface.

The temporary visual indicator, according to the present invention, can be a dye that imparts a color for a set period of time. As used herein, the term “color” includes colors of all shades, hues, and intensities visible to the naked or assisted eye. Color changes of an indicator may be triggered by a variety of physical or chemical reactions. The presence of certain gases, changes in H₂O concentrations (i.e., humidity, moisture level), changes in temperature, or exposure to UV light may all be used in methods to effect color changes of certain indicators.

Non-limiting examples of temporary visual indicators include those light unstable dyes disclosed in U.S. Pat. No. 5,548,010, such as Basonyl® green NB-832 (triarylmethane) (BASF Corp., Rensselaer, N.Y.), Basonyl® Blue-650 (triarylmethane) (BASF Corp., Rensselaer, N.Y.), “Flexo Yellow” 110 LD (diarylmethane) (BASF Corp., Rensselaer, Nyf); FD&C#2 Blue Powder (indigotene) (Hilton-Davis, Co., Cincinnati, Ohio), FD&C#2 AL. Lake, FD&C#2 (indigotene) (Triton Colors, Inc., Elmwood Park, N.J.), Pylam® Blue LX 5595 (triarylmethane) (Pylam Products Co., Inc., Garden City, N.Y.), FD&C Blue #1 Powder (triphenylmethane) (Warner-Jenkinson Co., Inc., St. Louis, Mo.), FD&C#1 Al.Lake (Warner-Jenkinson Co., Inc., St. Louis, Mo.), FD&C Yellow #5 (pyrozoine) (Wamer-Jenkinson Co., Inc., St. Louis, Mo.), FD&C Green #3 (triphenylmethane) (Warner-Jenkinson Co., Inc., St. Louis, Mo.), Erythrosine Lake #9301 (Warner-Jenkinson Co., Inc., St. Louis, Mo.), and FD&C Yellow Lake #5 (Warner-Jenkinson Co., Inc., St. Louis, Mo.).

Other suitable temporary visual indicators of the present invention may be found in U.S. Pat. No. 4,954,544. Phenolphthalein, 3,3-bis[4-hydroxyphenyl]-1-[3H]isobenzofuranone, is colorless in its lactone form, the form present in solutions below pH 8.5. In solutions above pH 9.0, the lactone form of the molecule loses two protons to form an intensely colored red dianion. Thus, materials formulated with the temporary visual indicator with a pH above 9.0 are colored pink, or red, by the phenolphthalein.

Other temporary visual indicators that are sensitive to pH levels include ascorbic acid & iron chloride, bromthymol blue, thymol, blue phenol red, cresol red, m-cresol purple, m-cresol red, methyl violet, methyl orange, bromocresol green, methyl red, thymolphthalein, and alizarin yellow. Bromothymol blue, thymol blue, m-cresol purple, m-cresol red, and phenol red are purple or blue under basic conditions and yellow under neutral conditions. Cresol red is purple under basic conditions and orange under normal conditions. Methyl violet and bromocresol green are yellow under acidic conditions and blue under normal conditions. Methyl orange and methyl red are both red under acidic conditions and yellow under normal conditions. Thymolphthalein is blue under basic conditions and colorless under normal conditions. Alizarin yellow is red under basic conditions and yellow under normal conditions.

In a related embodiment, a user can customize materials for application to a desired surface, wherein the materials include a temporary indicator that is visible at a first pH and invisible at a second pH. Materials having a temporary visible indicator are formulated by the user by mixing to a selected material a temporary visual indicator that is sensitive to pH and a volatile base (i.e., monoamines and diamines) or acid (i.e., hydrochloric acid or thionyl chloride). The volatile base or acid provides a means for a rapid change in the pH after application of the material to a surface. With a change in pH, the indicator can change from visible to invisible.

The volatile base or acid can be added to the material prior to, during, or after addition of the indicator. Alternatively, the indicator can be added to the material prior to or after the addition of the volatile base or acid. In addition, the volatile base or acid can be mixed with the indicator prior to addition to the selected material.

In a specific embodiment, a user customizes a material for application to a surface by selecting the material and phenolphthalein as the temporary visual indicator. The material and phenolphthalein are then mixed together with a volatile base to form a customized composition to be applied to a desired surface. The volatile base preferably raises the pH of the customized composition to above 9.0. At that pH, phenolphthalein is in its red dianion form and the customized composition is colored red or pink by the indicator. On application of the pink (or red) material to a desired surface, the volatile nature of the base causes the base to evaporate or degrades rapidly. As the base evaporates, the pH of the composition falls below pH 8.5, phenolphthalein returns to its colorless, lactone form and the composition is no longer colored by the indicator. A mild solution of the volatile base can later be sprayed onto the surface to which the composition was applied to temporarily raise the pH above 9.0 and reactivate the indicator to the red dianion form.

Volatile bases appropriate for use in the subject invention include, but are not limited to, monoamines such as ammonia, methyl amine, dimethylamine, trimethylamine, ethyl amine, isopropyl amine, butyl amine, pentyl amine, hexyl amine and octyl amine, diamines such as ethylene diamine, 1,2-diaminopropane, 1-3-diaminopropane and 1,2-diaminobutane or cyclic amines such as tetrahydropyrrole. Of the volatile bases listed above, ammonia and pentyl amine are currently used as inactive ingredients in marketed drug products.

Contemplated volatile acids appropriate for use in the subject invention include, but are not limited to, hydrochloric acid, thionyl chloride, acetic acid, lactic acid, citric acid, bromoacetic acid, 2-bromobutyric acid, 2-bromocaproic acid, 2-bromocaprylic acid, 2-bromohexanoic acid, 2-bromoisobutyric acid, 2-bromoisovaleric acid, 2-bromo-3methylbutyric acid, 2-bromo-2-methylpropionic acid, 2-bromomyristic acid, 2-bromooctanoic acid, 2-bromophenylacetic acid, 2-bromopropionic acid, 2-bromotetradecanoic acid, meso-2,3-dibromosuccinic acid, malic and tartaric, isobtyric, butyric, isovaleric, valeric, hexanoic, 3-methylvaleric, heptanoic, and nonanoic acids.

Although phenophthalein and a single volatile base are exemplified in the preferred embodiment, other temporary visual indicators, alone or in combination, and other physical and chemical reactions effecting a color change and providing a temporary visual indication of the location and/or concentration of the applied material are also embodied by this invention.

In certain instances, volatile bases or acids may be present in the material for application and only the indicator dye will need to be added. Reference to degradation or evaporation of a base or acid with regard to changing pH is directed to a chemical conversion or reaction. For example, in the case of a volatile base, a chemical conversion/reaction takes place when water absorbs carbon dioxide thereby reducing the OH⁻ concentration and, consequently, the pH level. With this example, the CO₂ may be supplied directly or simply absorbed from ambient air. In the case of a volatile acid, the acid deprotonates, thereby reducing the H⁺ concentration and, consequently, raising the pH.

In another embodiment, a user mixes a temporary visual indicator with medicinal compositions that are administered to a patient. This would provide the advantage of allowing the user to control the duration of time the indicator is detectable and/or the concentration of medication to be administered to the patient. Further, the resultant customized composition can be applied evenly and completely over the areas to be treated. Often times, medicinal compositions need to be applied at a certain thickness for efficacy. The color intensity of the indicator of the invention can vary with the amount of material applied. Thus, a customized composition comprising a temporary visual indicator and mediation component can be uniformly applied with an appropriate dosage/concentration. In one embodiment, a color guide is provided to the user, in which a color match system assures that an appropriate concentration/amount of the medicinal composition has been mixed with the temporary visual indicator.

In a further embodiment, a reactivating solution is provided, which can cause the indicator to be visually detectable. Application of the reactivating solution to an area on which the customized composition was applied enables the user to ascertain whether the material is still present. After a brief period of time, the indicator disappears so as not to interfere with the user's activity.

In accordance with the present invention, the individual user can mix a temporary visual indicator with topical materials. For example, the temporary visual indicator can be mixed with a surgical scrub. Accordingly, the user has the option of selecting a preferred surgical scrub to be applied to skin. By mixing the indicator with the preferred surgical scrub, the user is provided with a means for assessing whether an area has been adequately disinfected. Alternatively, a user can mix a temporary visual indicator with topical materials in order to indicate a set period of time of treatment with the topical materials. Thus, the user has the option of selecting a surgical scrub that is non-toxic to dermal cells, as opposed to iodine. Moreover, the temporary nature of the indicator of the invention ensures that no color is left on the skin to interfere with surgical marks to be used in a procedure.

As used herein, the term “topical materials” includes, and is not limited to, creams, sprays, lotions, gels, foams, emollients, waxes, pastes, milks, mousses, balms, scrubs, and the like. These materials may be used for a number of applications, including, but not limited to, hair (i.e., hair dyes), hand, facial, and body lotions; cold creams; facial or body moisturizers; anti-acne preparations; topical analgesics; cosmetics including foundations, eyeshadows, lipsticks, and the like; cleansers, toners; facial masks (i.e., firming, moisturizing, purifying, deep-cleansing); insect repellant formulations; deodorants; dusting powders; antiperspirants; depilatory creams; shaving products (i.e., shaving cream, gel, or foam); suncare products (i.e., sunscreens; sunblockers); after sun lotion, milk, and gel; burn lotion; tanning lotion; sunless self-tanning cream, spray, and lotion; combination sunscreen-insect repellant formulations; and mascara products (i.e., thickening, lengthening, waterproof).

For security situations, a user can mix a temporary visual indicator with a material that is applied to a surface area requiring surveillance. Because the indicator eventually becomes invisible, it is undetected by someone tampering with the surface. However, the indicator can be treated to become visible and show whether the surface has been disturbed. Further, the person tampering with the surface may pick up some of the indicator on his person or clothing, which can also be treated and made visible. Alternatively, an invisible hand stamp containing a temporary visual indicator, which becomes visible upon treatment, can serve as an alternative to present UV-visible technology.

The visual indicators of the subject invention can be used to improve the utility of a variety of product applications in addition to those described above. For example, the accuracy of a spray is often unreliable. Therefore, a user can mix a temporary visual indicator with any spray product to make certain that the entire area is effectively covered by the spray. Non-limiting examples of sprays to which users can mix a temporary visible indicator include dermatological sprays such as antibacterial, insect-repellent, and medicinal; household sprays such as cleaning solutions and disinfectants; agricultural sprays such as fertilizer, funcicides, herbicides, and insecticide sprays.

A temporary visual indicator formulated with pesticides, herbicides or fertilizers ensures adequate treatment of all areas with the compound. Uneven greening of a plant caused by disproportionate coverage of the plant with a pesticide is avoided. The indicator formulated with a pesticide for in-home use does not stain floors, walls or woodwork to which the pesticide is applied. The presence of the indicator not only ensures that the pesticide is adequately applied, but also ensures that the pesticide is accurately applied. The color guide provided by the temporary indicator guarantees that children's toys, plants or pet dishes lying on the floor are not mistakenly sprayed with the pesticide. Pots, pans, utensils, and food items within cabinets and drawers being treated will likewise be protected from the effects of stray pesticide. Items that are mistakenly sprayed are immediately identifiable and can be washed to remove the pesticide. The volatile base can be applied to the washed item to verify no residual pesticide is present.

Herbicides formulated with the temporary visual indicator allow for selective application of the product to plants. The herbicide N-phosphomethyl glycine, (glyphosate), is a broad-spectrum, non-selective herbicide that kills virtually all vegetation it contacts. Selective application is essential. The temporary visual indicator formulated with this herbicide provides a color guide for application of the herbicide so that only those plants that need to be treated are treated and that treated plants are fully sprayed. Herbicides applied in the wind or under conditions created by a fan in a greenhouse are accurately applied when formulated with the temporary visual indicator of the subject invention. The indicator formulated in cropdusting compounds will ensure the delivery of the herbicide is complete and on target. Fertilizers formulated with a temporary visual indicator ensures that the soil to which they are applied is adequately covered.

To assess whether a particular area has been effectively treated, a user can mix a temporary visible indicator with such materials as dental sealants, to ensure that the entire tooth is covered and adequately sealed; and ophthalmic solutions, to verify adequate administration of the solution to the eye.

To assess whether proper and complete coverage of a surface has been accomplished for decorative or structural surfaces, a user can mix a temporary indicator with paints, varnishes, or lacquers. When applying a second coat of paint or other similar material, the temporary visual indicator ensures that fresh paint is applied to the entire painted surface so that paint will not dry unevenly or blotchy. In working with clear finishes such as lacquers or varnishes, it is often difficult to tell which areas have or have not been covered. The temporary indicator provides a color guide while the finish is being applied which rapidly disappears so that the clear finish properties of the compound are retained. Car polish or other polishes formulated with a temporary visual indicator ensure complete coverage of the car with the polish, but do not stain or discolor the finish of the car. A user can also mix a temporary visual indicator with a grease compound to not only identify whether an area is adequately greased, but also to ensure that other surfaces are not soiled by the grease compound. Teflon sprays and coatings which are difficult to remove if misapplied are accurately applied when formulated with a temporary visual indicator. By enabling the user to mix a temporary visual indicator with a product of choice, the user is ensured that these products are accurately applied.

By mixing a temporary visual indicator with protective sealants, the user can know if an area is completely sealed and protected. Fabrics and carpets are often treated to protect against stains. These textiles sometimes have intricate weaves or deep naps and it is difficult to be sure that all areas of the fabric are adequately treated. A temporary visual indicator formulated in protective sprays provides the user with a color guide for applying the protectant so that the user is sure that even recessed areas of a weave are protected from stains. A temporary visual indicator formulated into sealants such as waterproofing agents for wooden decks allows the user to be sure that the deck is fully sealed. The temporary nature of the indicator, however, ensures that the sealant will not mask the grain or stain the wood.

When preparing customized materials for application to a surface, a sufficient amount of temporary visual indicator to provide an adequate visual signal is mixed with a selected material. In a preferred embodiment, the quantity of temporary visual indicator mixed with the selected material does not affect the nature of the material. Suitable compositions of the present invention contain not more than 10% weight and preferably from 0.001% to about 2% by weight of the indicator.

With embodiments in which the temporary visual indicator is a pH sensitive compound, the amount of volatile component (i.e., volatile base or acid) added is sufficient to change the pH of the entire composition to a level that will affect the indicator and be present in an amount sufficient to maintain the pH for an adequate time period after material application. Volatile components can be selected with respect to their rate of evaporation or degradation.

According to the subject invention, a device or kit or any other packaging system can be supplied to a user, wherein the kit comprises at least one compartment that includes a temporary visual indicator as defined above. In certain embodiments wherein the presence of a modifying substance is contemplated, the kit can include a second compartment that includes the modifying substance as defined above. These kits can be equipped with instructions for mixing the temporary visual indicator and/or modifying substance with a desired material to prepare a customized composition for application to a surface.

All patents, patent applications, provisional applications, and publications referred to or cited herein are incorporated by reference in their entirety, including all figures, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for making a customized composition for application to a surface wherein said method comprises selecting a material; selecting a temporary visual indicator; and mixing the indicator with the material to form the customized composition.

2. The method, according to claim 1, further comprising the step of mixing a modifying substance with the material, wherein the modifying substance enables the indicator to be visible for a desired period of time after application, and then disappears.

3. The method according to claim 2, wherein the indicator is a compound that is selected from the group consisting of phenolphthalein; bromthymol blue; thymol blue; phenol red; cresol red; m-cresol purple; methyl violet; methyl orange; bromocresol green; methyl red; thymolphthalein; and alizarin yellow.

4. The method according to claim 2, wherein the indicator is a compound that is visible at a first pH and invisible at a second pH.

5. The method according to claim 4, wherein the material is selected from the group consisting of herbicides, pesticides, fertilizers, protectants, sealants, cleansers, polishes, varnishes, lacquers, and topical materials.

6. The method according to 4, wherein the indicator is selected from the group consisting of 3,3-bis[4-hydroxyphenyl]-1-[3H]-isobenzofuranone and thymolphthalein.

7. The method according to claim 2, wherein the modifying substance is a volatile base or acid.

8. The method according to claim 7, wherein the modifying substance is selected from the group consisting of a monoamine, a diamine, a cyclic amine, hydrochloric acid, thionyl chloride, acetic, malic and tartaric, isobutyric, butyric, isovaleric, valeric, hexanoic, 3-methylvaleric, hetanoic, and nonanoic acids.

9. The method according to claim 1, wherein the indicator is a compound that is visible at a first temperature and invisible at a second temperature.

10. The method according to claim 1, wherein the indicator is selected from the group consisting of basonyl green; basonyl blue; diarylmethane; FD&C #2 indigotene; FD&C #2 lake; triarylmethane (pylam blue); FD&C #1 triphenylmethane; FD&C #1 lake; FD&C #5 yellow; pyrazoine; FD&C #3 green; tripheynylmethane; FD&C #3 red; erthyrosine powder; and FD&C #5 yellow lake.

11. The method according to claim 1, wherein the indicator is a compound that is visible at a first moisture level and invisible at a second moisture level.

12. The method according to claim 1, wherein the indicator is a compound that is visible prior to exposure to light and invisible after exposure to light.

13. The method according to claim 1, wherein the indicator is a compound that is visible prior to reaction with an oxidizing agent and invisible after reaction with an oxidizing agent.

14. The method according to claim 1, wherein the indicator is a compound that is visible prior to reaction with a reducing agent and invisible after reaction with a reducing agent.

15. The method according to claim 1, wherein the material is non-paint material.

16. A composition comprising:
(a) a non-paint material selected for application to a surface; and
(b) a temporary visual indicator that is initially visible and capable of becoming substantially invisible after application of the composition to a surface, wherein the temporary visual indicator is a light unstable dye.

17. The composition according to claim 16, wherein the material is selected from the group consisting of herbicides, pesticides, fertilizers, cleansers, and topical materials;

18. The composition according to claim 16, wherein the temporary visual indicator is selected from the group consisting of basonyl green; basonyl blue; diarylmethane; FD&C #2 indigotene; FD&C #2 lake; triarylmethane (pylam blue); FD&C #1 triphenylmethane; FD&C #1 lake; FD&C #5 yellow; pyrazoine; FD&C #3 green; tripheynylmethane; FD&C #3 red; erthyrosine powder; and FD&C #5 yellow lake.

19. A kit for making a customized composition for application to a surface, wherein said kit comprises at least one compartment that includes a temporary visual indicator, wherein the temporary visual indicator is initially visible and capable of becoming substantially invisible; and instructions for making the customized composition.

20. The kit according to claim 19, wherein said temporary visual indicator is a light unstable dye.

21. The kit according to claim 19, wherein the temporary visual indicator is selected from the group consisting of basonyl green; basonyl blue; diarylmethane; FD&C #2 indigotene; FD&C #2 lake; triarylmethane (pylam blue); FD&C #1 triphenylmethane; FD&C #1 lake; FD&C #5 yellow; pyrazoine; FD&C #3 green; tripheynylmethane; FD&C #3 red; erthyrosine powder; and FD&C #5 yellow lake.

22. The kit according to claim 19, further comprising a second compartment that includes a modifying substance, wherein the modifying substance enables the indicator to be visible for a period of time, wherein the period of time the indicator is visible is dependent on the modifying substance mixed with the material; and instructions for mixing the modifying substance.

23. The kit according to claim 22, wherein the indicator is selected from the group consisting of phenolphthalein; bromthymol blue; thymol blue; phenol red; cresol red; m-cresol purple; methyl violet; methyl orange; bromocresol green; methyl red; thymolphthalein; and alizarin yellow.

24. The kit according to claim 22, wherein the indicator is a compound that is visible at a first pH and invisible at a second pH.

25. The kit according to claim 24, wherein the indicator is selected from the group consisting of 3,3-bis[4-hydroxyphenyl]-1-[3H]-isobenzofuranone and thymolphthalein.

26. The kit according to claim 22, wherein the modifying substance is a volatile base or acid.

27. The kit according to claim 26, wherein the modifying substance is selected from the group consisting of a monoamine, a diamine, a cyclic amine, hydrochloric acid, thionyl chloride, acetic, malic and tartaric, isobutyric, butyric, isovaleric, valeric, hexanoic, 3-methylvaleric, hetanoic, and nonanoic acids.
